(19) United States
(12) Patent Application Publication
(10) Pub. No.: US 2017/0071586 A1
(43) Pub. Date: Mar. 16, 2017

(54) TONGUE RETRACTORS FOR FRENOTOMIES

(71) Applicant: The General Hospital Corporation, Boston, MA (US)

(72) Inventor: Marvin Wang, Boston, MA (US)

(21) Appl. No.: 15/123,456

(22) PCT Filed: Apr. 23, 2015
(86) PCT No.: PCT/US15/27322

§ 371 Date: Sep. 2, 2016

Related U.S. Application Data
(60) Provisional application No. 61/983,088, filed on Apr. 23, 2014.

(51) Int. Cl. A61B 13/00; A61B 17/24; A61B 17/02

(52) U.S. Cl. CPC A61B 13/00; A61B 17/02; A61B 17/244; A61B 2017/00814

(57) ABSTRACT

This disclosure provides tongue retractors for oral medical procedures such as frenotomies. The retractors include first and second planar members extending from a vertex, wherein the first and second members extend from the vertex at a predetermined angle so as to provide upward retraction of the tongue of a subject when the retractor is placed in the subject's mouth; and an opening within at least one of the members. The opening is arranged to expose the frenulum of the subject and to permit an operator to surgically operate on the frenulum while the retractor is placed in the subject's mouth. The disclosure also provides uses of the retractors in procedures to cut a frenelum, e.g., to treat ankyloglossia.

### CROSS-REFERENCE TO RELATED APPLICATION

This application claims the benefit of U.S. Provisional Application No. 61/983,088, filed on Apr. 23, 2014, the contents of which is incorporated herein by reference in its entirety.

### TECHNICAL FIELD

This invention relates to tongue retractors, and more particularly to tongue retractors for lingual frenotomies (also known as lingual frenectomies).

### BACKGROUND

Neonatal frenotomies are performed routinely in the newborn nursery as well as in the pediatricians' office to treat partial ankyloglossia (also called tongue-tie), which is caused by a frenum of the tongue that is abnormally short or is attached too close to the tip of the tongue. The procedure is typically done easily without any anesthesia, and the patient can be returned to the parents immediately after the procedure. However, as with many surgeries, frenotomy carries a potential risk of damage to the surrounding tissues. The current practice allows for physicians to cut the lingual frenulum freehand, while typically an assistant will pry the mouth open with a finger on each side of the mouth. At the same time, the assistant will attempt to retract the tongue with the same fingers that are prying open the mouth.

These issues allow for a great deal of potential movement on the typically unwilling patient's part, while the surgeon is attempting to cut a very fine membrane. In addition, because the physician typically does not have the cutting hand supported in any way, there is always a great potential to miss the target and damage the surrounding tissue structures.

The incidence of ankyloglossia has been described to exist in up to 10% of all newborns. Of those with ankyloglossia, 25% have been reported to have breast-feeding difficulties. Studies show that performing frenotomies in these children improve breastfeeding ability for all treated cases. However, there is a huge population, especially in the third world, which may not be able to breastfeed adequately, because of ankyloglossia. Giving these populations a resource to quickly and easily treat this problem would be a great aid for the health of breastfeeding infants worldwide (Ballard et al., “Ankyloglossia: assessment, incidence, and effect of frenuloplasty on the breastfeeding dyad,” Pediatrics, 110(5):e63 (2002)(see, the internet at “uptodate.com/contents/ankyloglossia-tongue-tie-in-infants-and-children”).

### SUMMARY

This disclosure relates to new tongue retractors and methods of use. In one aspect, this disclosure features tongue retractors for oral medical procedures. The new retractors include first and second planar members extending from a vertex, the first and second members extending from the vertex at a predetermined angle so as to provide upward retraction of the tongue of a subject when the retractor is placed in the subject's mouth; and an opening within at least one of the members, the opening arranged to expose the frenulum of the subject and to permit an operator to surgically operate on the frenulum while the retractor is placed in the subject's mouth.

In various embodiments the opening includes a slit extending through each of the two planar members, wherein the slit runs in parallel with a longitudinal axis of each member beginning at the vertex. In some embodiments the slit extends through at least about half of the length of each of the first and second members. For example, the width of the slit can be configured to fit a frenulum within the slit, e.g., the width of the slit can be about 2 to 10 mm, e.g., 3 to 7 mm, e.g., 3, 4, or 5 mm.

In certain embodiments, the predetermined angle is about 45 degrees, e.g., about 35 to 55 degrees. In different implementations the two planar members can be made of one elongate piece of metal or plastic folded or curved at the vertex to form the two planar members in a V-shape or U-shape or can be made of two elongate pieces of metal or plastic connected at the vertex to form a V-shape or U-shape.

In some implementations the tongue retractors described herein can further include a handle member extending from an end portion of one of the first and second planar members, wherein the handle member is arranged so that the retractor can be moved and manipulated by the operator when holding the handle member. The handle member can be an elongate post extending at an angle, e.g., an oblique angle such as about 100 to 130 degrees, relative to a plane of the planar member.

In various implementations, the of the tongue retractors described herein, the first and second planar members can be about 20 to 40 mm in length and 15 to 25 mm in width. The tongue retractors can consist or, or consist essentially of, materials suitable for use in an autoclave. In some implementations the planar members and/or the handle of the tongue retractor can be made of one or more metal and/or plastic materials. For example, the planar members can be made of plastic and the handle of metal, or vice versa. Alternatively, the entire device can be made of one or more metals or of one or more plastics.

In another aspect, the disclosure provides the tongue retractors described herein for use in frenotomies. These uses can include any one or more of the features of the tongue retractors recited and described herein.

In another aspect, the disclosure features methods of surgically operating on the frenulum of a subject. These methods include placing a tongue retractor as described herein in the mouth of a subject, e.g., under the tongue; retracting the tongue of the subject upwardly with the retractor; exposing the frenulum of the patient's tongue through the opening; and cutting the frenulum of the subject by way of surgical operation through the opening. The methods can include any one or more of the features of the tongue retractors recited and described herein.

The new tongue retractors provide excellent control of the tongue on newborn patients and users have indicated a significant improvement in control and safety of frenotomies when using the new retractors. These retractors can be used to push back the tongue and lower mouth, allowing exposure only of the frenulum. This, in turn, will allow the physician to cut the frenulum while knowing that the retractor is protecting the surrounding structures. In addition, the retractors allow the user to separate the tongue from the floor of the mouth and create a wide exposure of the frenulum, allowing the user to optimize the frenulum cut while safely protecting all surrounding mouth structures from being potentially accidentally cut. Thus, by keeping the planar members in front of the tongue and mouth structures, the new retractors prevent accidental cuts and complications from occurring during the procedure.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic perspective view of one embodiment of the new tongue retractor device with two planar members at an angle, e.g., about 45 degrees, from a vertex where they join, and a handle member extending from an end portion of one of the first two members.

FIGS. 2A and 2B are images of two different embodiments, one of metal (FIG. 2A) and one of a stiff plastic (FIG. 2B) of the new devices.

### DETAILED DESCRIPTION

In general, this disclosure provides tongue retractors for oral medical procedures.

Tongue Retractors

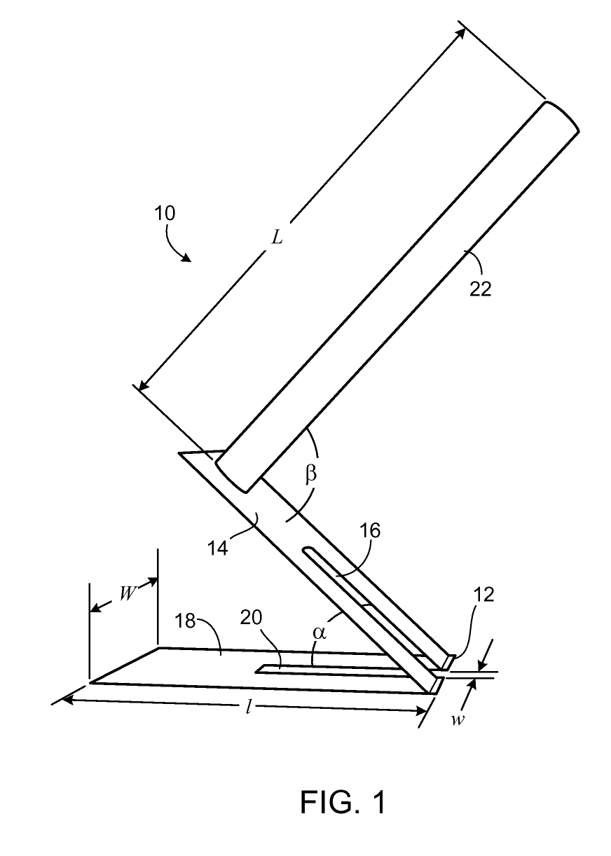

As shown in FIG. 1, the tongue retractor 10 includes two, flattened, planar members 14, 18, extending from a vertex 12 to form a “V.” The members 14, 18 extend from the vertex 12 at a predetermined angle α so as to provide upward retraction of the tongue of a subject when the retractor is placed in the subject's mouth. The retractor 10 includes an elongate opening or slit 16 and/or 20 within at least one of the members 14, 18, wherein the opening is arranged to enable the frenulum of the subject to fit into the slit and to permit an operator to surgically operate on the frenulum while the retractor is placed in the subject's mouth.

In some embodiments, the opening includes both a slit 16 extending through member 14 and slit 20 extending through member 18, wherein the two slits are located along a central axis of each member, proximate to and connecting at vertex 12.

In some embodiments, one or both slits extend through about half or more of the length of each of the members. In some embodiments, the width (w) of the slit is about 2 to 10 mm, e.g., 3 to 7 mm, e.g., 3, 4, or 5 mm. In some embodiments, the first and second members can be made of one piece of metal or plastic, and can be folded, e.g., in half, to form the two members on either side of the vertex. The vertex 12 can be a sharp curve to form a V-shape or a somewhat gentler curve to form a U-shape (still maintaining angle α), but the underlying goal is not to manufacture an apex that is sharp enough to cut into the tissue. The slit 16, 20 can be made through both halves of the single piece of metal or plastic when the two halves are folded together, or can be cut or stamped through the one piece before it is folded. Alternatively, the two members 14, 18 can be manufactured separately, and the slit cut into them. Then the two members can be connected, e.g., by soldering or gluing, at vertex 12. As noted above, the vertex should not be made sharp enough to cut into tissue. However, the vertex must also be sufficiently rigid to prevent the two planar members from flexing much with respect to each other, thus maintaining an adequate exposure of the surgical field.

In different embodiments, the predetermined angle α is about 30 to 60 degrees, e.g., about 35 to 55 degrees, 40 to 50 degrees, e.g., 43, 44, 45, 46, or 47 degrees.

In some embodiments, the tongue retractor further includes an elongate handle member 22 extending for a length (L) from an end portion of member 14 at an angle β. The handle member 22 is arranged so that the retractor 10 can be moved and manipulated by the user when holding the handle member. In some embodiments, the handle member 22 comprises an elongate post extending at angle β, which can be, for example, a right angle or an oblique angle, relative to the plane of member 14. This angle β can be about 80 to 140 degrees, e.g., about 80 to 120 degrees, 90 to 120 degrees, or about 90, 100, 110, or 120 degrees.

In different embodiments, each of the two flattened planar members 14, 18 are about 20 to 40 mm in length (l), e.g., 25 to 35 m, e.g., 30 mm in length and 15 to 25 mm, e.g., 20 mm in width (W).

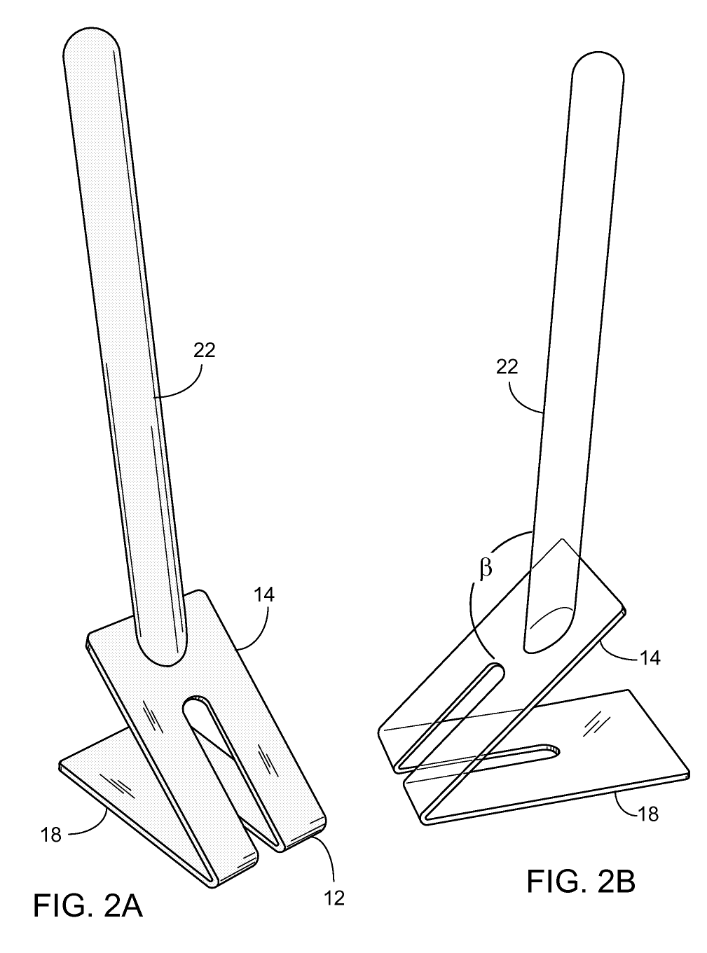

As shown in FIG. 2A, the tongue retractors can be constructed of materials suitable for use in an autoclave, such as certain metals, e.g., stainless steel, titanium, or alloys, e.g., titanium or cobalt alloys. Alternatively, as shown in FIG. 2B, the tongue retractors can also be constructed of plastic, such as polyethers, polyetherimides, polyphenylenes, polyphenylsulfones, nylons, polyphenylsulfones, polycarbonates, and polyesters. These can be self-reinforced, or reinforced with glass fibers or particles or carbon fibers or particles, and can be suitable for disposal after a single use.

In all embodiments and for both metal and plastic materials, the new tongue retractors can be sterilized using conventional sterilization methods, including autoclaving, gamma radiation, ethylene oxide, boiling, and steam. For metal tongue retractors, they can be sterilized and used repeatedly. The plastic retractors can be initially sterilized, but can then be disposed of after a single use.

Methods of Use of the Tongue Retractors

This disclosure also provides new methods for surgically operating on the frenulum of a subject. In general, the methods include placing a tongue retractor as described herein in the mouth of the subject so that the tongue of the subject is retracted upwardly. The tongue retractor further includes an opening or slit within at least one of the members that is arranged so that the frenulum of the subject is exposed. The method includes cutting the frenulum of the subject by way of surgical operation through the opening.

More specifically, the infant is first swaddled to keep the hands away from the surgical area. An assistant opens the corners of the mouth as wide as possible with his or her fingers. The user then places the vertex of the device into the mouth, keeping the handle above the planar members, in front of the patient's face. The vertex of the planar members is then pushed backwards between the bottom of the tongue and the floor of the mouth, allowing the lingual frenulum to slide into the middle slit. The user continues to push the retractor back fully to the blind end of the mouth where the tongue meets the floor of the mouth, maximizing the exposure and extension of the lingual frenulum. At this point, the user will use a pair of surgical scissors appropriate for the infant size, and cut the frenulum. If an adequate amount of frenulum has been cut, the retractor is removed from mouth, and the infant is returned to the mother to reinitiate breastfeeding. If the frenulum was not adequately cut, the retractor stays in place to allow the user to continue cutting as needed.

The following example does not limit the scope of the claims.

### Example

The new tongue retractors have been used experimentally in 34 newborn patients (e.g., on average they are 2 to 3 days old). The new retractors fit well into the mouth of all of these patients, and also properly exposed the frenulum in all of these patients. The stability and protection of the mouth was assessed by the users on a scale of 1 to 5 (with 5 being the best) and the overall assessment for the 34 cases was 4.97. There were no complications in any of the patients.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A tongue retractor for oral medical procedures comprising:
first and second planar members extending from a vertex, the first and second members extending from the vertex at a predetermined angle so as to provide upward retraction of the tongue of a subject when the retractor is placed in the subject's mouth; and
an opening within at least one of the members, the opening arranged to expose the frenulum of the subject and to permit an operator to surgically operate on the frenulum while the retractor is placed in the subject's mouth.

2. The tongue retractor of claim 1, wherein the opening comprises a slit extending through each of the two planar members, wherein the slit runs in parallel with a longitudinal axis of each member beginning at the vertex.

3. The tongue retractor of claim 2, wherein the slit extends through at least about half of the length of each of the first and second members.

4. The tongue retractor of claim 2, wherein the width of the slit is configured to fit a frenulum within the slit.

5. The tongue retractor of claim 1, wherein the predetermined angle is about 45 degrees.

6. The tongue retractor of claim 1, wherein the two planar members are made of one elongate piece of metal or plastic folded or curved at the vertex to form the two planar members in a V-shape or U-shape.

7. The tongue retractor of claim 1, wherein the two planar members are made of two elongate pieces of metal or plastic connected at the vertex to form a V-shape or U-shape.

8. The tongue retractor of claim 1, further comprising a handle member extending from an end portion of one of the first and second planar members, wherein the handle member is arranged so that the retractor can be moved and manipulated by the operator when holding the handle member.

9. The tongue retractor of claim 8, wherein the handle member comprises an elongate post extending at an angle relative to a plane of the planar member.

10. The tongue retractor of claim 9, wherein the angle is an oblique angle.

11. The tongue retractor of claim 10, wherein the oblique angle is about 100 to 130 degrees.

12. The tongue retractor of claim 1, wherein each of the first and second planar members are about 20 to 40 mm in length and 15 to 25 mm in width.

13. The tongue retractor of claim 1, wherein the tongue retractor consists of materials suitable for use in an autoclave.

14. The tongue retractor of claim 1, wherein the planar members of the tongue retractor comprise one or more metal materials.

15. The tongue retractor of claim 1, wherein the planar members of the tongue retractor comprises one or more plastic materials.

16. The tongue retractor of claim 1, wherein the handle member comprises one or more metal materials.

17. The tongue retractor of claim 1, wherein the handle member comprises one or more plastic materials.

18. A tongue retractor of claim 1 for use in frenotomies.

19. A method of surgically operating on the frenulum of a subject, the method comprising:
placing a tongue retractor of claim 1 in the mouth of a subject;
retracting the tongue of the subject upwardly with the retractor;
exposing the frenulum of the patient's tongue through the opening; and
cutting the frenulum of the subject by way of surgical operation through the opening.
